(19) United States
(12) Patent Application Publication
(10) Pub. No.: US 2013/0041432 A1
(43) Pub. Date: Feb. 14, 2013

(54) DEVICE FOR PROMOTING GROWTH OF EYEBROW HAIR

(71) Applicants: Gavin Tucker, Irvine, CA (US); Nicholas Brox, Laguna Beach, CA (US); Jeffrey Braile, Boca Raton, FL (US); Morgan Pepitone, Newport Beach, CA (US)

(72) Inventors: Gavin Tucker, Irvine, CA (US); Nicholas Brox, Laguna Beach, CA (US); Jeffrey Braile, Boca Raton, FL (US); Morgan Pepitone, Newport Beach, CA (US)

(21) Appl. No.: 13/569,813

(22) Filed: Aug. 8, 2012

Related U.S. Application Data
(60) Provisional application No. 61/521,002, filed on Aug. 8, 2011.

(51) Int. Cl. A61N 5/06; A61N 5/067

(52) U.S. Cl. 607/89; 607/90

(57) ABSTRACT

A device for promoting eyebrow hair growth includes an array of light generating sources, such as LEDs, laser diodes and IPLs, which are housed within a brow plate, for providing evenly distributed light to a user's eyebrows at a low level output wavelength suitable for stimulating hair growth. This photo-biostimulation process promotes hair growth in the directed region by producing an increase in ATP and keratin production, enhancement in blood flow and circulation, as well as an increase in collagen production. Temple arm members with corresponding ear pieces or a headband are used to support the device on a user's head. The light generating sources may be powered by an internal power source, such as a rechargeable battery or disposable batteries, located within the headband or temple arm members, or by an external power source, such as a plug used in connection with an AC outlet.

### BACKGROUND OF THE INVENTION

This non-provisional application is based on U.S. provisional patent application serial no. 61/521,002 filed on Aug. 8, 2011.

1. Field of the Invention

This invention relates to eyebrow hair treatment and, more particularly, to a device that combines light generating sources with a headband or sunglasses supporting device, capable of promoting growth of a user's eyebrow hair by way of evenly distributed light of low level output wavelengths directed onto a user's eyebrows.

2. Discussion of the Related Art

Loss of eyebrow hair is common among men and women. In many instances, thinning of the eyebrows can be attributed to regular removal of eyebrow hair, which is particularly common for women who pluck their eyebrows for aesthetic purposes. Eyebrow thinning may also result from include excessive plucking and rubbing resulting from a nervous habit, or medical conditions wherein patients experience eyebrow hair loss as a side effect of treatment.

In response, several products and methods have been developed for treating or masking an individual's eyebrow hair loss, including topical solutions that promote hair growth and tattoos for filling in the thinning areas of the eyebrows. However, application of a topical solution can require a particularly arduous process that is not always effective, while the tattooing process can be painful and the results are not realistic in appearance. The present invention seeks to address these considerable drawbacks of the presently available products and methods for treating eyebrow hair loss.

The present invention provides for the application of phototherapy to the eyebrows to prevent hair loss and re-grow new hair. Phototherapy consists of exposure to specific wavelengths of light using lasers, light emitting diodes (LED's) (both individual and arrays), IPL's (Intense Pulsed Light) and other light sources, for a prescribed amount of time to both treat disease and affect cosmetic enhancements to the hair, scalp and skin. The use of phototherapy in medical science and aesthetics is rapidly evolving as more and more wavelengths of light are being identified to target various sections of cells in order to stimulate cellular proficiency and enhance the body's ability to heal and rejuvenate itself.

### OBJECTS AND ADVANTAGES OF THE INVENTION

Considering the foregoing, it is a primary object of the present invention to provide a device that utilizes phototherapy for promoting growth of eyebrow hair.

It is a further object of the present invention to provide an eyebrow hair growth promotion device that directs evenly distributed light at low level output wavelengths towards a user's eyebrows.

It is a further object of the present invention to provide an eyebrow hair growth promotion device that is wearable and hands-free.

It is a further object of the present invention to provide an eyebrow hair growth promotion device that is easy to use and does not require application of a topical solution.

These and other objects and advantages of the present invention are readily apparent with reference to the detailed description and accompanying drawings.

### SUMMARY OF THE INVENTION

The present invention is directed to a wearable hands-free device that provides phototherapy treatment by directing an array of light generating sources, which are housed within a brow plate, towards a user's eyebrows for providing evenly distributed light to the user's eyebrows. For this application, the phrase “light generating sources” includes, but is not limited to, light emitting diodes (LEDs), laser diodes, infrared, and intense pulse lights (IPLs). The light generating sources emit light at a low level output wavelength suitable for stimulating hair growth. This photo-biostimulation process promotes hair growth in the directed region by producing an increase in ATP and keratin production, enhancement in blood flow and circulation, as well as an increase in collagen production. The light generating sources may be powered by an internal power source, such as a rechargeable battery or disposable batteries, located within the headband or temple arm members, or by an external power source, such as a plug used in connection with an AC outlet.

### BRIEF DESCRIPTION OF THE DRAWINGS

For a fuller understanding of the nature of the present invention, reference should be made to the following detailed description taken in conjunction with the accompanying drawings in which:

FIG. 1 is a top plan view of the eyebrow hair growth promotion device of the present invention, in accordance with one embodiment, illustrating an array of light generating sources housed within two brow plates and supported by a nose piece, temple arm members and ear piece configured to wrap around a user's ears;

FIG. 2 is a side profile view of the eyebrow hair growth promotion device of FIG. 1 illustrating light blocking visors provided for shielding a user's eyes from the light generating sources;

FIG. 3 is a perspective view illustrating the interior side of the eyebrow hair growth promotion device of FIG. 1, showing the array of light generating sources located on the inner facing side of two brow plates, which are supported by a nose piece, temple arm members and ear pieces configured to wrap around a user's head;

FIG. 4 is a side profile view of the eyebrow hair growth promotion device of FIG. 1 illustrating the array of light generating sources in spaced, opposing relation to a user's eyebrows;

FIG. 5 is a front view illustrating the eyebrow hair growth promotion device of FIG. 1 being worn by a user; and

FIG. 6 is a side profile view of the eyebrow hair growth promotion device, shown in accordance with another embodiment, illustrating an array of light generating sources located on the inner facing side of two brow plates, which are supported by a headband configured to wrap around a user's head.

Like reference numerals refer to like referenced parts throughout the several views of the drawings.

### DETAILED DESCRIPTION OF THE PREFERRED EMBODIMENTS

Referring to the several views of the drawings, the wearable hands-free device for promoting eyebrow hair growth is shown in accordance with multiple embodiments and is generally indicated as 10.

In each of the embodiments of the invention, the eyebrow hair growth promotion device 10 includes brow plates 12 that house an array of light generating sources 14, such as light emitting diodes (LEDs), laser diodes, infrared, intense pulse lights (IPLs), or other suitable light sources that are adapted to emit evenly distributed light within a wavelength suitable for promoting hair growth (e.g., 620-700 nm). Each brow plate 12 further includes a light blocking visor 16 located beneath the light generating sources 14 and is provided for shielding light from the user's eyes. The light blocking visors 16 may be comprised of a rigid material or a flexible material that is either hingedly attached or fixed to the brow plate 12.

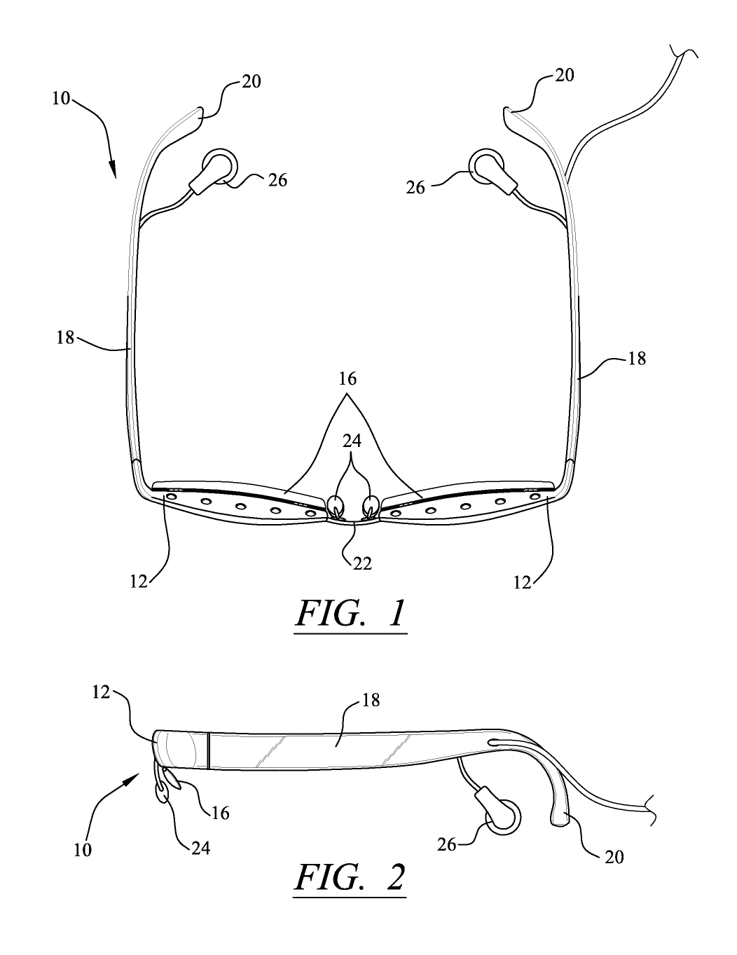

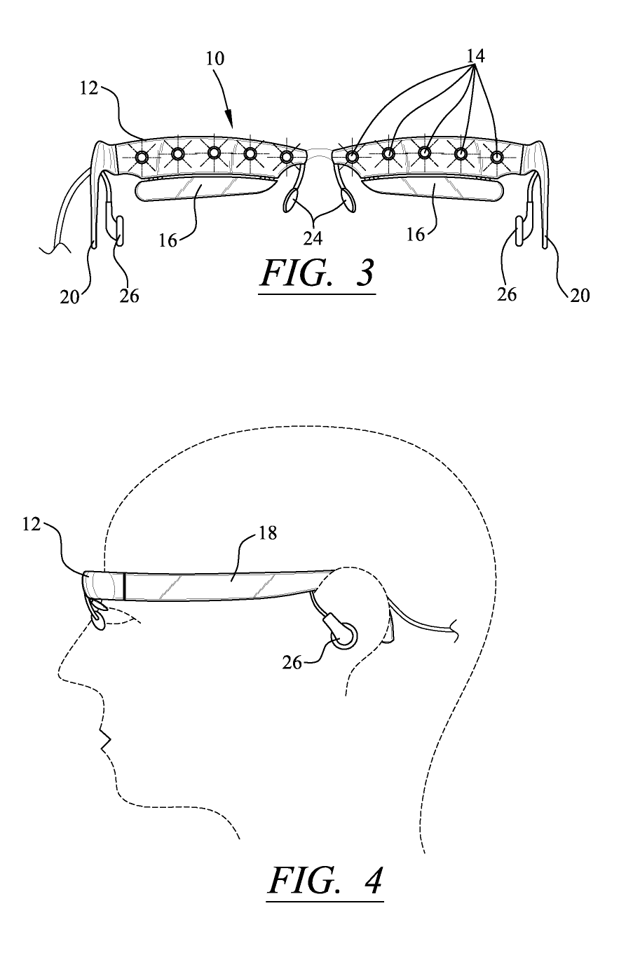

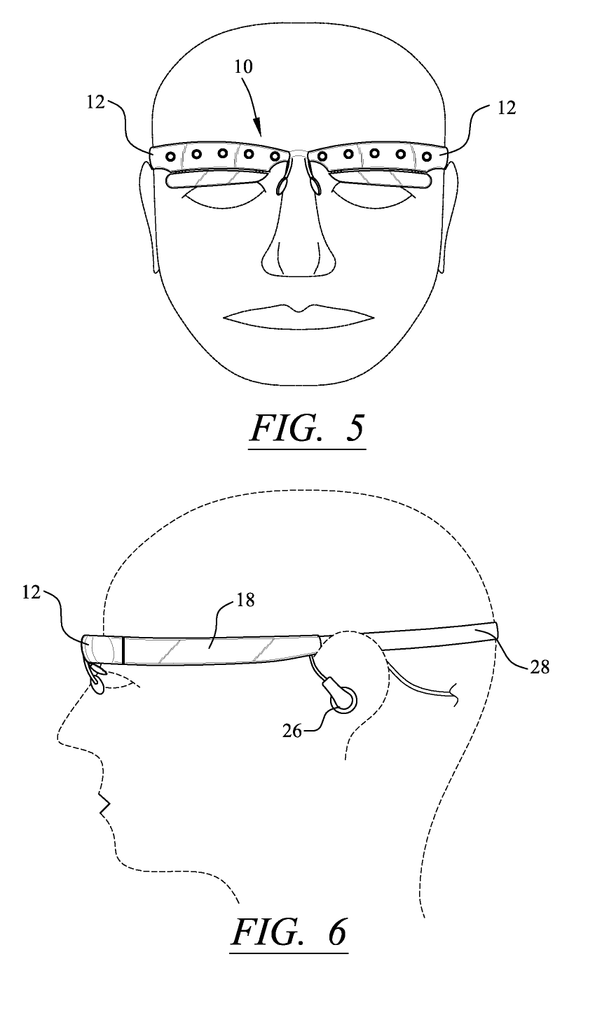

A preferred embodiment of the eyebrow hair growth promotion device 10 is illustrated in FIGS. 1-5 and utilizes temple arm members 18 and corresponding ear pieces 20 that are configured to wrap around the user's ears, similar to a pair of glasses, for supporting the brow plates 12 such that the light generating sources 14 are positioned in spaced, opposing relation to the user's eyebrows. A noise bridge 22 is included for providing additional support about the user's nose. Nose pads 24 are included for allowing the eyebrow hair growth promotion device 10 to rest comfortably on the user's nose. Earphones 26 may be integrally connected to the temple arm members 18 or ear pieces 20 for use in conjunction with a portable audio player or, alternatively, the temple arm members 18 may include a portable media player and earphones 26 for storing and playing audio files.

An alternate preferred embodiment of the eyebrow hair growth promotion device 10 is illustrated in FIG. 6, wherein a headband 28 is used to support the brow plates 12 such that the light generating sources 14 are positioned in spaced, opposing relation to the user's eyebrows. The headband 28 is made out of an elastomeric material and is configured for expanding to wrap around the user's head and retracting to conform to the contour of the user's head. The headband 28 may extend from the brow plates 12 or, alternatively, from temple arm members 18, as shown in FIG. 6. The alternate embodiment may include the nose bridge 22 and nose pads 24, as well as earphones 26 for use in conjunction with a portable media player, which may be integrally connected to the headband 28.

The eyebrow hair growth promotion device 10 may be powered by an internal power source, such as a rechargeable battery or disposable batteries, located within the headband 28, temple arm members 18 or ear pieces 20, or by an external power source, such as a plug used in connection with an AC outlet. The user controls the device 10 using a set of controls, which may be included on the device 10 or on a remote control system.

In operation, the light generating sources 14 direct evenly distributed light towards a user's eyebrows at low level output wavelengths, which penetrates the skin to a depth suitable for stimulating hair growth. Cell absorption of the low level output wavelengths generates an increase in ATP and keratin production, enhancement in blood flow and circulation, as well as an increase in collagen production.

While the invention has been shown and described in accordance with several preferred and practical embodiments thereof, it is recognized that departures from the instant disclosure of the invention are fully contemplated within the spirit and scope of the invention and such changes, variations and modifications of the present invention are not to be limited except as recited in the following claims as interpreted under the Doctrine of Equivalents.

What is claimed is:

1. A wearable hands-free device for promoting growth of eyebrow hair on a user, said device comprising:
a first and a second brow plate each including an inner side;
a first temple arm member extending from said first brow plate and a second temple arm member extending from said second brow plate, and said first and second temple arm members being structured and disposed for supporting said device on the user's head with said first and second brow plates positioned in spaced, opposing relation to the user's eyebrows and defining an operable position;
an array of light generating sources on said inner side of said first and second brow plates, and said array of light generating sources being positioned, structured and disposed for producing a light pattern that is simultaneously directed onto the user's eyebrows when said device is in the operable position, and each of said light generating sources being further structured and disposed for emitting light within a wavelength range for promoting hair growth; and
at least one control for controlling operation of each of said array of light generating sources.

2. The device as recited in claim 1 further comprising a first ear piece extending from said first temple arm member and a second ear piece extending from said second temple arm member, and said first and second ear pieces being sized and configured for wrapping partially around the user's ears.

3. The device as recited in claim 1 further comprising an elastomeric headband having a first end and a second end, the first end being coupled with the first temple arm member and the second end being coupled with the second temple arm member, and said elastomeric headband being sized and configured for wrapping around a back portion of the user's head and conforming to the shape of the user's head.

4. The device as recited in claim 1 wherein said light generating sources are adapted for emitting light within a wavelength range of 620 nm-700 nm.

5. The device as recited in claim 1 wherein said light generating sources are light emitting diodes (LEDs).

6. The device as recited in claim 1 wherein said light generating sources are laser diodes.

7. The device as recited in claim 1 wherein said light generating sources are intense pulse lights (IPLs).

8. The device as recited in claim 1 wherein said light generating sources are infrared lights.

9. The device as recited in claim 1 wherein said at least one control is located on the device.

10. The device as recited in claim 1 wherein said at least one control is a remote control.

11. The device as recited in claim 1 further comprising a pair of audio emitting earphones supported on said first and second temple arms, respectively, and positionable on the user's ears.

12. A wearable hands-free device for promoting growth of eyebrow hair on a user, said device comprising:
a first and a second brow plate each including an inner side, and said inner sides being positionable in spaced, opposing relation to the user's eyebrows when said device is worn in an operable position on the user's head;
an elastomeric headband having a first end and a second end, the first end being coupled with said first brow plate and the second end being coupled with said second brow plate, said elastomeric headband being sized and configured for wrapping around at least a portion of the user's head and conforming to the shape of the user's head to support said device in the operable position;
an array of light generating sources on said inner side of said first and second brow plates, and said array of light generating sources being positioned, structured and disposed for producing a light pattern that is simultaneously directed onto the user's eyebrows when said device is worn in the operable position, and each of said light generating sources being further structured and disposed for emitting light within a wavelength range for promoting hair growth; and
at least one control for controlling operation of each of said array of light generating sources.

13. The device as recited in claim 12 wherein said light generating sources are adapted for emitting light within a wavelength range of 620 nm-700 nm.

14. The device as recited in claim 12 wherein said light generating sources are light emitting diodes (LEDs).

15. The device as recited in claim 12 wherein said light generating sources are laser diodes.

16. The device as recited in claim 12 wherein said light generating sources are intense pulse lights (IPLs).

17. The device as recited in claim 12 wherein said light generating sources are infrared lights.

18. The device as recited in claim 12 wherein said at least one control is located on the device.

19. The device as recited in claim 12 wherein said at least one control is a remote control.

20. The device as recited in claim 12 further comprising a pair of audio emitting earphones positionable on the user's ears when said device is worn in the operable position.
